(19) United States
(12) Patent Application Publication
(10) Pub. No.: US 2006/0199276 A1
(43) Pub. Date: Sep. 7, 2006

(54) DEVICE FOR MULTIPLE RAPID DETECTION AND TESTING OF DISEASES, DISORDERS AND IMMUNITY IN HUMAN, ANIMALS AND THE ENVIRONMENT

(76) Inventor: Bernhard Armani, Vancouver (CA)

Correspondence Address: VERMETTE & CO., BOX 40, GRANVILLE SQUARE, SUITE 230 - 200 GRANVILLE STREET, VANCOUVER, BC, V6C 1S4, CA

(21) Appl. No.: 10/906,798

(22) Filed: Mar. 7, 2005

(51) Int. Cl. G01N 33/558

(52) U.S. Cl. 436/514

(57) ABSTRACT

According to the invention there is provided a device for testing for diseases disorders and immunity in humans, animals and the environment, which includes a bottom membrane support assembly having a lower opening and operative to receive a bottom membrane extending over said lower opening and a top membrane support assembly having an upper opening and operative to receive a top membrane extending across said upper opening. The top membrane support assembly is positionable over the bottom membrane support assembly with the upper opening aligned with the lower openings

### SUMMARY OF THE INVENTION

According to the invention there is provided a device for testing for diseases, disorders and immunity in humans, animals and the environment, which includes a bottom membrane support assembly having a lower opening and operative to receive a bottom membrane extending over said lower opening and a top membrane support assembly having an upper opening and operative to receive a top membrane extending across said upper opening. The top membrane support assembly is able to be positioned over the bottom membrane support assembly with the upper opening aligned with the lower opening.

The top and bottom membranes are aligned and parallel.

An absorbent pad may be located below the bottom membrane to absorb the various liquids that are poured over the membranes. Needless to say, the membranes are water permeable.

The bottom membrane support assembly may include a frame, an absorbent pad within said frame, and a bottom membrane mask plate having an opening, the opening positioned to overlay a membrane placed on the absorbent pad.

The top membrane support assembly may include an upper section, a top membrane support plate juxtaposed to a bottom of the upper section, and a lower section, the upper section, the top membrane support plate and the lower section all having aligned openings, the upper section able to be positioned to overlay a top membrane which extends across the opening in both the upper section and the top membrane support plate.

The lower section has an opening in one side to permit sliding over and away from the bottom membrane support assembly.

The lower section may be hinged at one end to a hinge plate.

In another aspect of the invention there is provided a method of testing for diseases and disorders, which includes providing spaced apart water permeable, parallel top and bottom membranes having deposited thereon desired antibodies or antigens and a protein marker, diluting a drop of sample liquid with a first liquid diluent, pouring the first diluent and dissolved sample over the top membrane and after being filtered by the top membrane, over the bottom membrane and pouring a second liquid over said top membrane, and after being filtered by the top membrane, over the bottom membrane, to render visible any spots resulting from reaction with the sample by antigens, antibodies, and/or protein on the top and bottom membranes.

The bottom membrane may be used as a control.

The membranes may be treated in localized areas with antigens and/or antibodies corresponding to up to 4 diseases or disorders.

The sample liquid may be a single drop of human blood.

### DETAILED DESCRIPTION WITH REFERENCE TO THE DRAWINGS

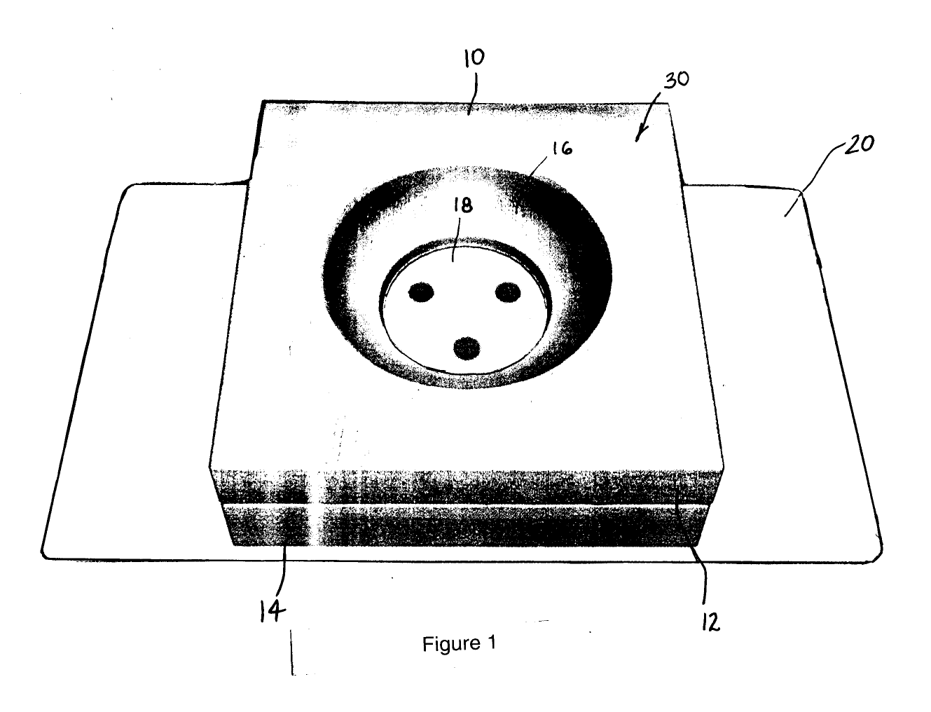

Referring to FIGS. 1-3, the testing device 10 has a top membrane support assembly 30 having an upper section 12 and a lower section 14. The upper section has a bowl-shaped recess 16 enclosing a circular opening 18. Below the upper section is a top membrane support plate 22 having an opening 24 identical in size to and, when assembled, aligned with opening 18. At each corner there are holes 27 which are dimensioned to receive legs 20 extending down from upper section 12. Lower section 14 has a lower section plate 25, which also has a large opening 26 of the same diameter as openings 24 and 18 and is aligned with the latter openings when assembled. Lower section plate 25 also has holes 28 at each corner equal to in diameter and aligned with corresponding holes 27.

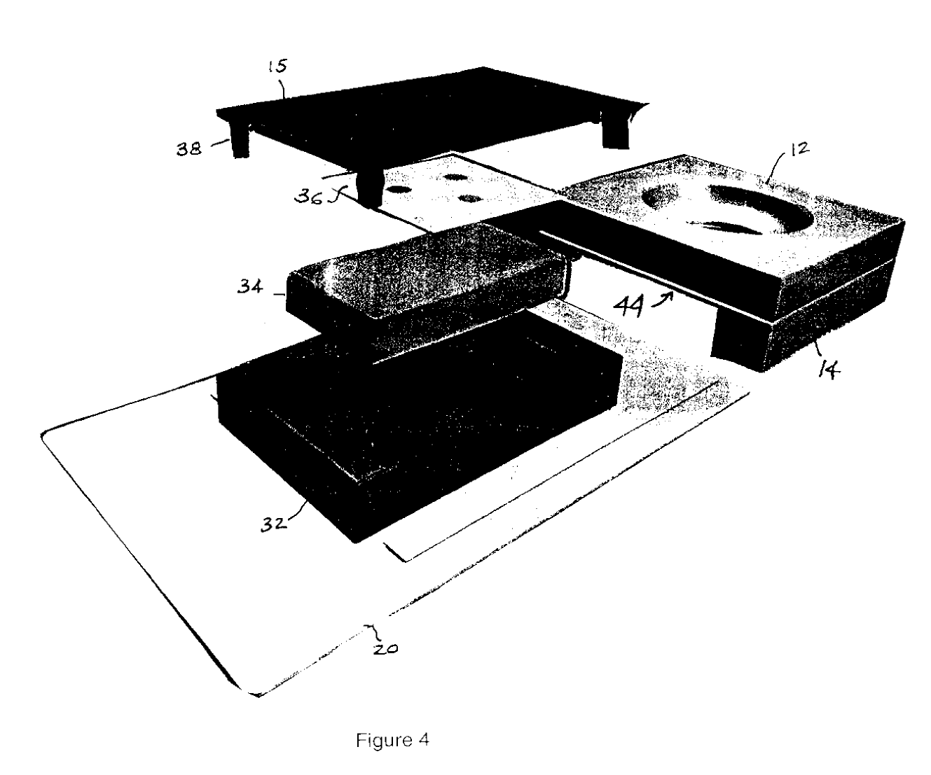

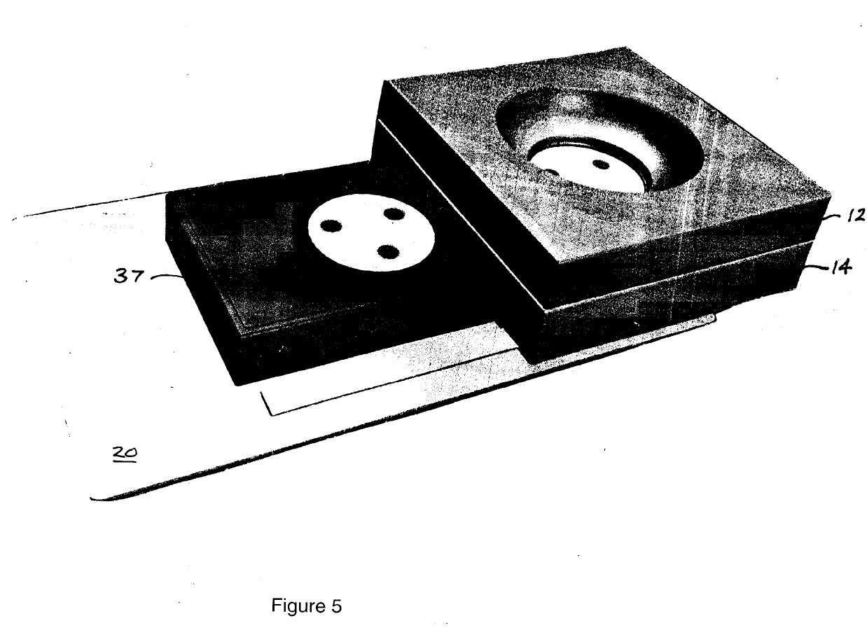

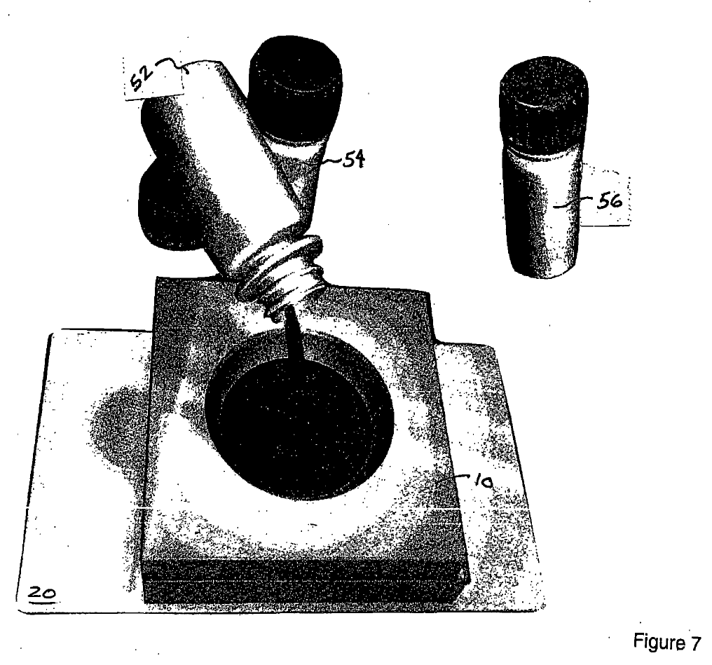

In FIGS. 1-9, reference number 20 identifies a support plate on which the device 10 rests. The support plate 20 allows the lower section 14 to slide over the bottom membrane section 37 as shown in FIGS. 4 and 5.

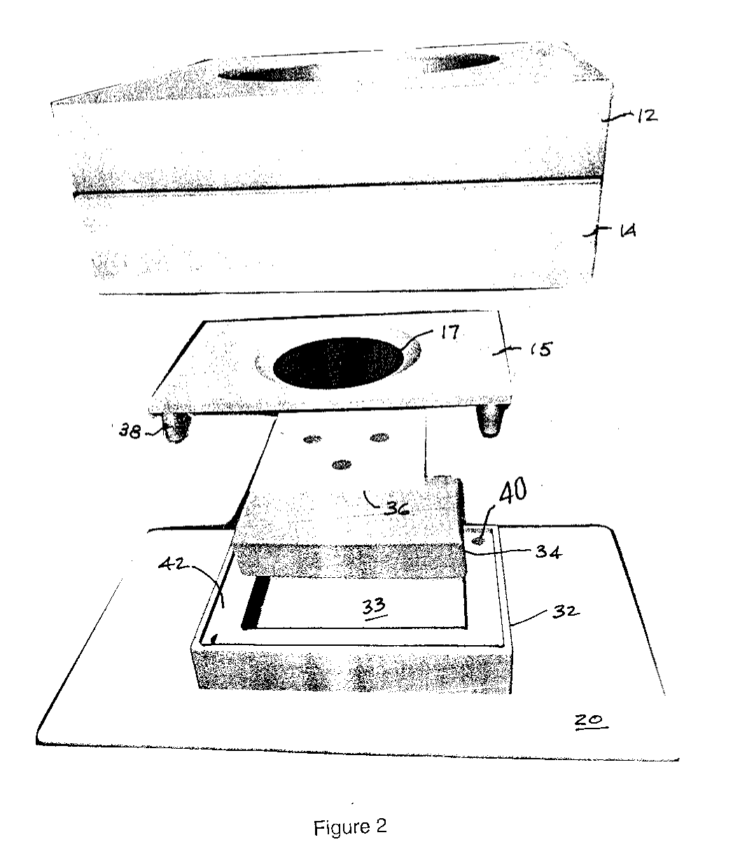

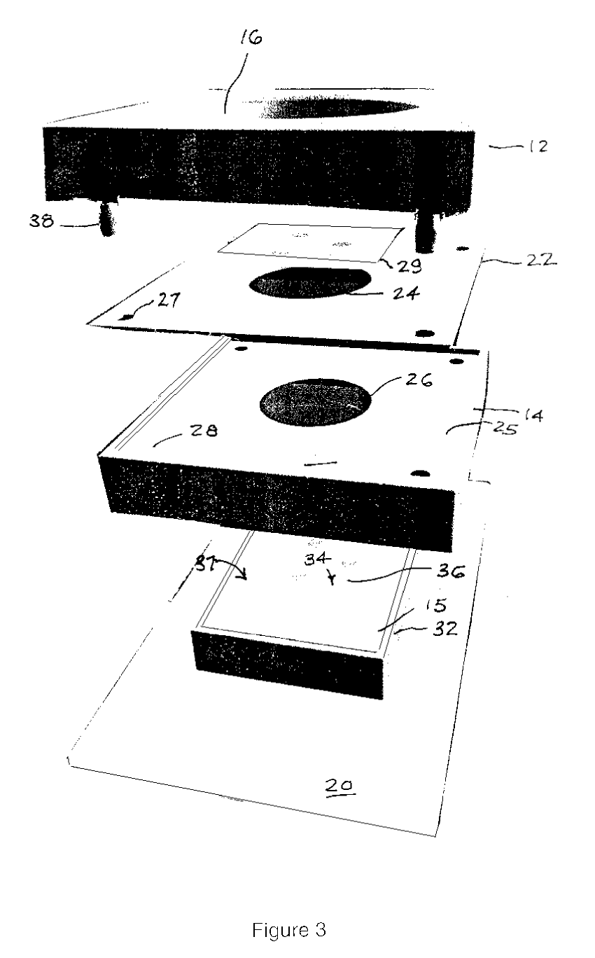

As seen in FIGS. 2 and 3 a bottom membrane support assembly 37 has a rectangular opening 33 in a rectangular frame 32 having a ledge 42 around an interior periphery thereof, said rectangular opening 33 which receives an absorbent cotton inside of a plastic cartridge 34 and, on top of the absorbent cotton 34, a lower synthetic top membrane 36. Legs 38 of a bottom membrane mask plate 15 having a central opening 17 with the legs at each corner that pass through holes 40 and a bottom membrane mask plate 15 that rests atop ledge 42 and overlies top membrane 36 and absorbent cotton 34. The assembled bottom membrane section 37 is seen in FIG. 3.

Referring to FIG. 4, the rectangular frame 32, the absorbent cotton 34, the top membrane 36, and the bottom membrane mask plate 15 are shown in exploded form adjacent to the lower section 14, which has an opening 44 into which slides the bottom membrane section 37. This can be seen more clearly in FIG. 5.

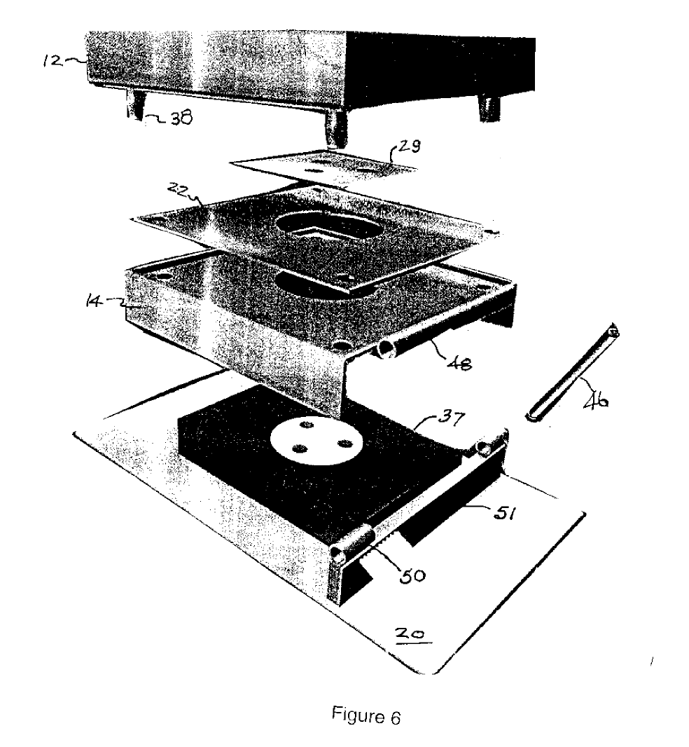

An alternate embodiment having a hinge is shown in FIG. 6. In this case a hinge sleeve 48 is integrally attached to an upper edge of plate 14. A pair of spaced apart hinge sleeve portions 50 are integrally attached to an upper edge of a hinge plate 51. With sleeve 48 aligned with sleeve portions 50, hinge pin 46 slides through the sleeve portions 50 and sleeve 48 locking the three together in a hinged connection. The remaining parts are the same as for the two embodiments discussed above. The device 10 is made of plastic both for ease of manufacture and cost.

Referring to FIGS. 6-9, in use the device 10 is assembled with the top synthetic membrane 29 and the bottom synthetic membrane 36 in place. Each membrane 29 and 36 has deposited on it in a given location either the desired antibodies or antigens for the test. The deposit on the synthetic membranes 29 and 36 are antibodies as well as a control being a protein-treated spot capable of capturing IgG antibodies normally present in blood and blood components. First a single drop of blood is transferred to the vial 52 which contains an inert diluent. Next the diluted blood sample is poured into the test device 10 where it reacts with the antibodies both on the top membrane 29 and on the bottom membrane 36 in this case producing circular dots or coloured regions. The control being a protein sensitive only to human blood of the person being tested reacts with the protein to produce a circular dot. If both the top membrane 29 and the bottom membrane 36 both show a control spot then the test is valid. In the event that any of the other areas of the membranes 29 and 36 react to form a visible spot then the person being tested tests positive for whichever disease or disorder is being tested. If the top and bottom membranes 29 and 36 are treated with antibodies or antigens corresponding to the same disease or disorder then the fact that spots show up on both membranes lends redundancy to the results and a consequent high reliability. Ordinarily the lower top membrane 36 is used as the control unit.

Alternatively, the top membrane 29 and bottom 36 membrane can be treated with antibodies or antigens corresponding to different diseases and/or disorders. In this case there can be up to four different diseases and/or disorders tested at one time. For example, the synthetic membranes 29 and 36 may be treated with antigens or antibodies corresponding to hepatitis A, hepatitis B and Avian Flu (e.g., H5 antigen).

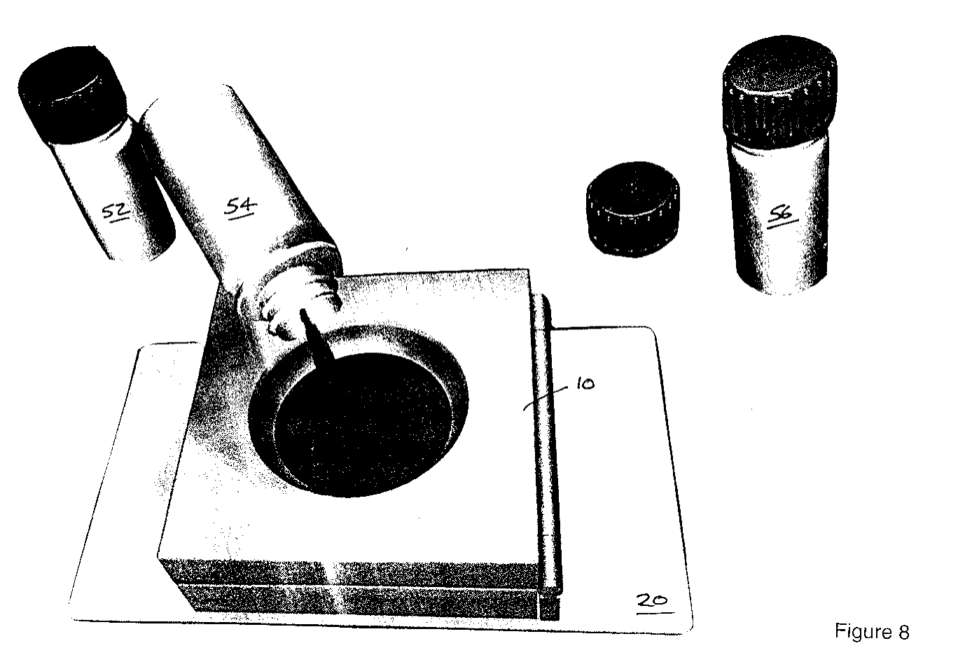

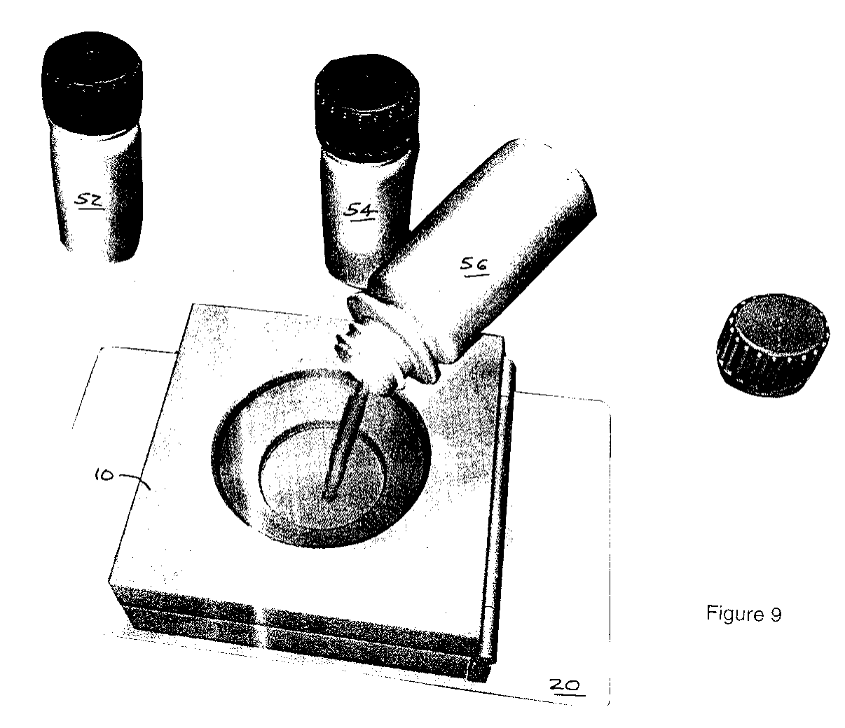

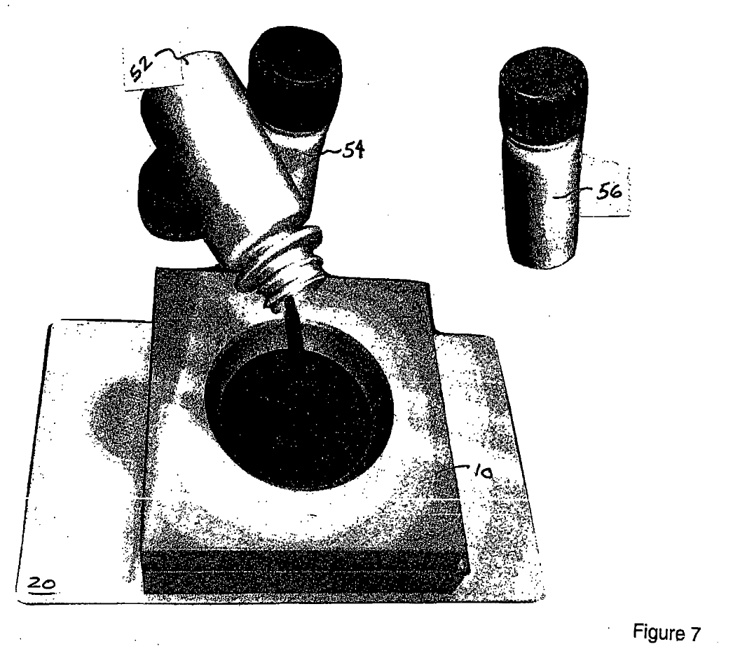

Referring to FIG. 8, the second step is to pour the second liquid in vial 54 over the top and bottom membranes 29 and 36, respectively. This liquid causes the spots to become visible. Referring to FIG. 9, the final vial 56 is poured over the top and bottom membranes 29 and 36, respectively, to cleanse the surfaces of the membranes for a better showing of the spots. Both liquids are known in the industry.

Accordingly, while this invention has been described with reference to illustrative embodiments, this description is not intended to be construed in a limiting sense. Various modifications of the illustrative embodiments, as well as other embodiments of the invention, will be apparent to persons skilled in the art upon reference to this description. It is therefore contemplated that the appended claims will cover any such modifications or embodiments as fall within the true scope of the invention.

1. A device for testing for diseases, disorders and immunity in humans, animals and the environment, comprising:
(a) a bottom membrane support assembly having a lower opening and operative to receive a bottom membrane extending over said lower opening; and
(b) a top membrane support assembly having an upper opening and operative to receive a top membrane extending across said upper opening;
wherein said bottom membrane and said top membrane are in vertically aligned and spaced relation to one another; and
wherein said bottom membrane and said top membrane are liquid permeable such that a liquid poured over said top membrane passes through said top membrane and said bottom membrane while said membranes are in said spaced relation to one another.

2. A device according to claim 1, wherein said top and bottom membranes are treatable with antibodies and antigens.

3. A device according to claim 1, wherein said top and bottom membranes are parallel.

4. A device according to claim 1, including an absorbent pad located below said bottom membrane.

5. A device according to claim 1, wherein said bottom membrane support assembly includes a frame, an absorbent pad within said frame, and a bottom membrane mask plate having an opening, said opening positioned to overlay a membrane placed on said absorbent pad.

6. A device according to claim 1, wherein said top membrane support assembly includes an upper section, a top membrane support plate juxtaposed to a bottom of said upper section, and a lower section, said upper section, said top membrane support plate and said lower section all having aligned openings, said upper section positionable to overlay a top membrane which extends across the opening in both said upper section and said top membrane support plate.

7. A device according to claim 6, wherein said lower section has an opening in one side to permit sliding over and away from said bottom membrane support assembly.

8. A device according to claim 6, wherein said lower section is hinged at one end to a hinge plate.

9. A device for testing for diseases and disorders in humans, animals and the environment, comprising:
(a) a bottom membrane support assembly having a lower opening and a bottom membrane extending across said lower opening; and
(b) a top membrane support assembly having an upper opening and a top membrane extending across said upper opening, said top membrane support assembly positionable over said bottom membrane support assembly with said upper opening aligned with said lower opening.

10. A device for testing for diseases, disorders and immunity in humans, animals and the environment, comprising:
(a) a bottom membrane support assembly having a lower opening and a bottom membrane extending across said lower opening; and
(b) a top membrane support assembly having an upper opening and a top membrane extending across said upper opening, said top membrane support assembly positionable over said bottom membrane support assembly with said upper opening aligned with said lower opening;
(c) a first liquid to dilute a sample;
(d) a second liquid to make spots visible; and
(e) a third liquid operative to clean up said membranes in regions outside of any spots.

11. A method of testing for diseases and disorders and immunity, comprising:
(a) providing spaced apart water permeable, parallel top and bottom membranes having deposited thereon desired antibodies or antigens and a protein marker;
(b) diluting a drop of sample liquid with a first liquid diluent;
(c) pouring said first diluent and dissolved sample over said top membrane and after being filtered by said top membrane, over said bottom membrane; and
(b) pouring a second liquid over said top membrane, and after being filtered by said top membrane, over said bottom membrane, to render visible any spots resulting from reaction with said sample by antigens, antibodies, and/or protein on said top and bottom membranes.

12. A method according to claim 11, wherein said bottom membrane is used as a control.

13. A method according to claim 11, wherein said membranes are treated in localized areas with antigens and/or antibodies corresponding to up to 4 diseases or disorders.

14. A method according to claim 11, wherein said sample liquid is a single drop of human blood.
